(19) United States
(12) Patent Application Publication
(10) Pub. No.: US 2002/0034556 A1
(43) Pub. Date: Mar. 21, 2002

(54) Insect repellent compositions

(76) Inventor: Donna Khazan, North Brunswick, NJ (US)

Correspondence Address: PENNIE AND EDMONDS, 1155 AVENUE OF THE AMERICAS, NEW YORK, NY, 100362711

(21) Appl. No.: 09/874,361

(22) Filed: Jun. 5, 2001

Related U.S. Application Data
(60) Provisional application No. 60/209,346, filed on Jun. 5, 2000.

(51) Int. Cl. A61K 35/78; A01N 25/00

(52) U.S. Cl. 424/725; 424/742; 424/747; 424/405

(57) ABSTRACT

The present invention details compositions for use in repelling insects that are inexpensive to manufacture, easily applied and/or integrated and non-injurious to plant and animal life, including citronella oil, geranium oil, rosemary oil, peppermint oil, D-limonene, aldehyde C-14 and aldehyde C-18. Preferred compositions additionally contain lavender oil, piperitone, and eucalyptus oil.

Additionally, the present invention details methods for repelling insects from a person, animal or physical structure by applying or integrating a composition described herein. The present invention further details articles that have been impregnated with insect repelling compositions described herein such as, for example, candles, tablecloths, inks, textiles and personal care products.

### TECHNICAL FIELD

The present invention relates to insect repellent compositions containing essential oils and two aldehydes. These compositions can be formulated to be oil soluble, cationic aqueous soluble and anionic aqueous soluble depending upon the intended use for the compositions. Additionally, the present invention relates to articles of manufacture such as, for example, candles, tablecloths, inks, textiles and personal care products impregnated with the insect repellent compositions described herein.

### BACKGROUND OF THE INVENTION

Insects breathe by means of tubes that open at the body surface in spiracles. The tubes divide into very fine branches leading to all the organs. The spiracles are water repellent, but oil may enter through them. Thus, many of the insect repellents known in the art contain essential oils. Regrettably, most of the known insecticides contain chemical compositions which, in certain concentrations, are harmful to the environment. Several researchers have attempted to produce insect repellents that do not contain chemicals harmful to the environment.

Citronella oil is a well-known and widely used insect repellent. The primary component responsible for its repellent properties is β-citronellol, 3,7-dimethyl-6-octen-1-ol, which is a member of the class of compounds known as the terpenes. J. L. Simonsen and L. N. Owen, The Terpenes Vol. 1, University Press, Cambridge, 2^{nd} ed., 1947.

D-Limonene is another insect repellent, although not as well-known and widely used as citronella oil. D-Limonene, 1-methyl-4-(1-methylethenyl)cyclohexene, is also a member of the terpene class. D-Limonen was first isolated from mandarin peel oil by Kugler and Kovats, Helv. Chim. Acta, 1963, 46, 1480.

U.S. Pat. No. 5,565,208 teaches compositions containing d-limonene, di-N-propyl isocinchomeronate, N-octyl bicycloheptene dicarboximide, C-11 alcohol ethoxylate, polysorbate 80, and an organic solvent selected from the group consisting of dipropylene glycol, N-butyl ether, citronella oil, dimethyl phthalate and N,N-diethyl-m-toluamide

U.S. Pat. No. 5,556,881 teaches compositions containing aqueous acetic acid and at least one essential oil. Examples of these essential oils include the following: spearmint oil, peppermint oil, oil of anise, sesame oil, oil of lemon, olive oil and mint extract.

To date, compositions that exhibit long-term repellent characteristics, which do not contain chemicals harmful to the environment have not been available. With the present concern over such insect-borne diseases as lyme disease carried by ticks and West Nile-like virus carried by mosquitoes, a distinct need exists for an environmentally friendly, effective insect repellent.

### SUMMARY OF THE INVENTION

An object of this invention is to provide compositions for use in repelling insects that are inexpensive to manufacture, easily applied and/or integrated and non-injurious to plant and animal life. The compositions contain citronella oil, geranium oil, rosemary oil, and peppermint oil, D-limonene, aldehyde C-14 (Dihydro-5-heptyl-2(3H)-furanone, CAS No. 104-67-6), and aldehyde C-18 (Dihydro -5-pentyl-2(3H)-furanone, CAS No. 104-61-0). Preferred compositions additionally contain lavender oil, piperitone or eucalyptus oil. The most preferred compositions contain one or more of the following fragrance compounds: spearmint oil, iso-bornyl acetate, vanillin, anethole, heliotropine, pine needle oil, aldehyde-C-10 (decyl aldehyde), lynalool, citral, lemon oil, grapefruit oil, N-amyl alcohol, lavandin oil, spike lavander, oil petitgrain mandarin, orange terpines, or neroli oil. These compositions can be formulated to be oil soluble, cationic water soluble or anionic water soluble depending upon the desired use of the compositions.

Another object of this invention is to provide methods for repelling insects from a person, animal or physical structure, comprising the following steps: formulating a composition of matter, including citronella oil, geranium oil, rosemary oil, peppermint oil, D-limonene, aldehyde C-14, and aldehyde C-18, such that the composition may be applied to a person, animal or physical structure or integrated into a physical structure; and applying or integrating the formulated composition to same. Preferred compositions additionally contain lavender oil, piperitone or eucalyptus oil. The most preferred compositions contain one or more of the following fragrance compounds: spearmint oil, iso-bornyl acetate, vanillin, anethole, heliotropine, pine needle oil, aldehyde-C-10, lynalool, citral, lemon oil, grapefruit oil, N-amyl alcohol, lavandin oil, spike lavander, oil petitgrain mandarin, orange terpines, or neroli oil.

Another object of the present invention is to provide articles that have been impregnated with the insect repellent compositions described herein such that the articles release the compositions thereby creating an insect free area around the article. Some example articles are candles, tablecloths, inks, textiles and personal care products.

### DETAILED DESCRIPTION OF THE INVENTION

The present invention relates to novel methods, compositions and articles manufactured utilizing these compositions that act as insect repellents. The compositions of the present invention are easily manufactured and do not contain any substances known to be harmful to the environment. Additionally, the compositions of the present invention may be formulated for use in different manufacturing processes to produce articles having insect repellent properties.

The compositions of the present invention comprise mixtures of citronella oil, geranium oil, rosemary oil, peppermint oil, D-limonene, aldehyde C-14, and aldehyde C-18.

The individual components of the compositions of the present invention are readily available commercially. The compositions of the present invention may be prepared to be oil soluble, cationic water soluble and anionic water soluble depending upon the desired use for the composition.

The practice of the invention is illustrated by the following examples, which are not to be considered limiting:

### EXAMPLE 1

To a standard laboratory beaker (500 ml) equipped with a magnetic stirring bar was introduced powdered vanillin (1.40 g), heliotropine (0.29 g) and anethole (0.29 g) were added to orange terpines (41.93 g) and stirred until the powders were completely dissolved. The remaining components from Table I were then added to the stirring solution to prepare the desired oil soluble composition. The relative percentages of the components of the composition are represented by weight in Table I.

TABLE I

| Component | Weight Percent |
|---|---|
| citronella oil | 3.25 |
| d-limonene | 4.54 |
| aldehyde C-14 | 0.40 |
| aldehyde C-18 | 0.39 |
| lavender oil | 4.17 |
| spearmint oil | 3.47 |
| peppermint oil | 1.16 |
| iso-bornyl acetate | 0.95 |
| geranium oil | 4.16 |
| vanillin | 1.40 |
| anethole | 0.29 |
| heliotropine | 0.29 |
| pine needle oil | 2.24 |
| aldehyde C-b | 0.59 |
| lynalool | 2.68 |
| rosemary oil | 0.76 |
| citral | 1.13 |
| piperitone | 0.10 |
| lemon oil | 4.39 |
| grapefruit oil | 4.46 |
| N-amyl alcohol | 0.06 |
| lavandin oil | 8.58 |
| spike lavander | 2.19 |
| oil petitgrain mandarin | 1.50 |
| eucalyptus oil | 0.31 |
| orange terpines | 41.93 |
| terpineol | 4.41 |
| neroli oil | 0.20 |

The resulting oil soluble composition had a pale yellow color and a specific gravity at 25° C. of 0.8580 g/ml. Additionally, the refractive index of the composition at 20° C. was 1.4720 and it exhibited a peak absorbance of 0.420 at 345 nm. The flash point of the composition was determined to be 130° F.

### EXAMPLE 2

The cationic water soluble composition corresponding to the composition of Example 1 was prepared using the same weight percentages of the components with the exception that the amount of orange terpines was reduced to 39.93 weight percent. The remaining 2.00 weight percent was made up by the addition of Tween 80 (polysorbate 80) solution.

The resulting cationic water soluble composition had a pale yellow color and a specific gravity at 25° C. of 0.8523 g/ml. Additionally, the refractive index of the composition at 20° C. was 1.4713 and it exhibited a peak absorbance of 0.375 at 345 nm. The flash point of the composition was determined to be 115° F.

### EXAMPLE 3

The anionic water soluble composition corresponding to the composition of Example 1 was prepared using the same weight percentages of the components with the exception that the amount of orange terpines was reduced to 39.93 weight percent. The remaining 2.00 weight percent was made up by the addition of sodium lauryl sulfate.

To a standard laboratory beaker (500 ml) equipped with a magnetic stirring bar was introduced powdered vanillin (1.40 g), heliotropine (0.29 g) and anethole (0.29 g) were added to orange terpines (39.93 g) and stirred until the powders were completely dissolved. The remaining components, with the exception of the sodium lauryl sulfate, were then added to the stirring solution. A portion of the resulting mixture, approximately 25% by volume, was removed and the powdered sodium lauryl sulfate was added to this portion with stirring. After the sodium lauryl sulfate was completely dissolved the portion containing the sodium lauryl sulfate was added back into the composition and mixed to yield the anionic water soluble composition.

The resulting anionic water soluble composition had a pale yellow color and a specific gravity at 25° C. of 0.8523 g/ml. Additionally, the refractive index of the composition at 20° C. was 1.4713 and it exhibited a peak absorbance of 0.375 at 345 nm. The flash point of the composition was determined to be 115° F.

### EXAMPLE 4

The composition of Example 1 was applied to the exposed forearm of a human subject and the forearm placed inside an enclosed space filled with ordinary house flies. The number of landings by flies upon the forearm was recorded and this number was compared to the number or flies that landed upon another subject's forearm that had been treated with a similar concentration of N,N-diethyl-m-toluamide (“DEET”). Fewer flies landed on the forearm treated with the composition of Example 1 than landed on the forearm treated with DEET for the same period of time.

### EXAMPLE5

The composition of Example 1 was tested against a similar concentration of DEET as described in Example 4 with the exception that mosquitoes were in the enclosed space instead of house flies. Fewer mosquitoes landed on the forearm treated with the composition of Example 1 than landed on the forearm treated with the DEET for the same period of time.

### EXAMPLE 6

The composition of Example 1 was homogenously mixed with molten paraffin wax in an amount equal to 3% by weight and poured into a mold containing a standard candle wick. When burned the candle was found to repel insects for the entire life of the candle.

### EXAMPLE 7

The composition of Example 3 was homogenously mixed into a paper slurry in an amount equal to 6% by weight before the slurry was pressed into sheets. The resulting sheets were formed into disposable, paper tablecloths. The tablecloths were found to repel insects for a period of 24 hours. The tablecloths were found to have an extended shelf life if stored in standard plastic packaging until the time of use.

### EXAMPLE 8

The composition of Example 2 was homogenously mixed into a screen printing ink in an amount equal to 6% by weight. The insect repellent ink was then used to make screen printed T-shirts. The T-shirts were found to repel insects for an extended period of time and after several washings. The T-shirts were found to have an extended shelf life if stored in standard plastic packaging until the time of use.

From the foregoing description, one of ordinary skill in the art can easily ascertain the essential characteristics of this invention and, without departing from its spirit or scope, can make various changes and modifications in the invention to adapt it to various usages and conditions.

1. An insect repellent composition, comprising: citronella oil; geranium oil; rosemary oil; peppermint oil; D-limonene; aldehyde C-14; and aldehyde C-18.

2. The composition of claim 1, further comprising lavender oil.

3. The composition of claim 2, further comprising piperitone.

4. The composition of claim 3, further comprising eucalyptus oil.

5. The composition of claim 5, further comprising at least one fragrance compound selected from the group consisting of spearmint oil, iso-bornyl acetate, vanillin, anethole, heliotropine, pine needle oil, aldehyde-C-10, lynalool, citral, lemon oil, grapefruit oil, N-amyl alcohol, lavandin oil, spike lavander, oil petitgrain mandarin, orange terpines, terpineol and neroli oil.

6. The composition of any one of claims 1 to 5, further comprising a surfactant to make the composition water soluble.

7. A method for repelling insects comprising the following steps:
a. formulating a composition of matter, comprising: citronella oil; geranium oil; rosemary oil; peppermint oil; D-limonene; aldehyde C-14; and aldehyde C-18, such that the composition may be applied to a person, animal or physical structure; and
b. applying the formulated composition of matter to a person, animal or physical structure in an amount sufficient to repel insects from the person, animal or physical structure.

8. The method according to claim 7, wherein the composition further comprises lavender oil.

9. The method according to claim 8, wherein the composition further comprises piperitone.

10. The method according to claim 9, wherein the composition further comprises eucalyptus oil.

11. The method according to claim 10, wherein the composition further comprises at least one fragrance compound selected from the group consisting of spearmint oil, iso-bornyl acetate, vanillin, anethole, heliotropine, pine needle oil, aldehyde-C-10, lynalool, citral, lemon oil, grapefruit oil, N-amyl alcohol, lavandin oil, spike lavander, oil petitgrain mandarin, orange terpines, terpineol and neroli oil.

12. An article of manufacture impregnated with an insect repellent composition, comprising:
citronella oil; geranium oil; rosemary oil; peppermint oil; D-limonene; aldehyde C-14; aldehyde C-18; lavender oil; piperitone; eucalyptus oil; spearmint oil; iso-bornyl acetate; vanillin; anethole; heliotropine; pine needle oil; aldehyde-C-10; lynalool; citral; lemon oil; grapefruit oil; N-amyl alcohol; lavandin oil; spike lavander; oil petitgrain mandarin; orange terpines; terpineol and neroli oil,
wherein the insect repellent composition is homogenously mixed throughout the article.

13. The article of claim 12, wherein the article is a candle capable of repelling insects when burnt.

14. The article of claim 12, wherein the composition further comprises a surfactant to make the composition water soluble.

15. The article of claim 14, wherein the article is a paper tablecloth capable of repelling insects.

16. The article of claim 14, wherein the article is an ink that may be used in screen printing textiles that is capable of repelling insects.
